(19) United States
(12) Patent Application Publication
(10) Pub. No.: US 2003/0162763 A1
(43) Pub. Date: Aug. 28, 2003

(54) Novel cephalosporin compounds and process for preparing the same

(76) Inventors: Chang-Seok Lee, Taejeon (KR); Seong-Ho Oh, Taejeon (KR); Eun-Jung Ryu, Taejeon (KR); Seong-Baek Lee, Taejeon (KR); Ha-Sik Youn, Taejeon (KR); Yong-Jin Jang, Taejeon (KR); Geun-Tae Kim, Taejeon (KR); Hyang-Sook Lee, Taejeon (KR)

Correspondence Address: BIRCH STEWART KOLASCH & BIRCH, PO BOX 747, FALLS CHURCH, VA, 22040-0747, US

(21) Appl. No.: 10/296,048

(22) PCT Filed: Jun. 14, 2001
(86) PCT No.: PCT/KR01/01026

(30) Foreign Application Priority Data
Jul. 7, 2000 (KR) 2000/38817

(51) Int. Cl. A61K 31/545; C07D 501/14

(52) U.S. Cl. 514/204; 514/205; 540/226; 540/227

(57) ABSTRACT

The present invention relates to a novel cephalosporin compound and pharmaceutically acceptable non-toxic salt, physiologically hydrolysable ester, hydrate, solvate or isomer thereof, to a pharmaceutical composition containing the compound and to a process for preparing the compound.

### TECHNICAL FIELD

The present invention relates to a novel cephalosporin compound useful as an antibiotic agent. More specifically, the present invention relates to a novel cephalosporin compound represented by the following formula (I), which is useful as an antibacterial agent, and particularly, exhibits a potent activity against strains such as methicillin-resistant Staphylococcus aureus (MRSA):

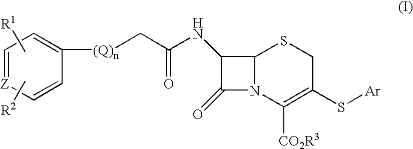

and pharmaceutically acceptable non-toxic salt, physiologically hydrolyzsable ester, hydrate, solvate or isomer thereof, in which

R¹ and R² independently of one another represent hydrogen, halogen, C₁₋₆ alkyl, C₁₋₆ alkylthio, aryl, arylthio, or C₅₋₆ heteroaryl containing one or two hetero atoms selected from the group consisting of nitrogen atom and oxygen atom;

R³ represents hydrogen or a carboxy-protecting group;

Q represents O, S or CH₂;

Z represents CH or N;

n denotes an integer of 0 or 1;

Ar represents a heteroaryl group represented by one of the following formulas:

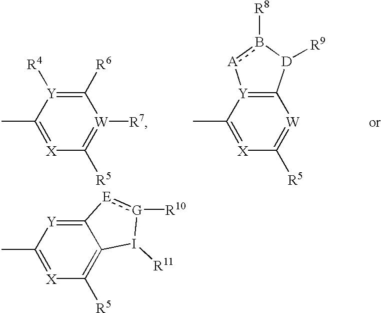

wherein X, Y, W, A, B, D, E, G and I independently of one another represent N or C (or CH), provided that the six-membered ring forms a pyrimidine structure;

R⁴ represents hydrogen or C₁₋₄ alkyl, or amino substituted or unsubstituted with a substituent selected from the group consisting of C₁₋₆ alkyl and C₁₋₆ hydroxyalkyl;

R⁵ and R⁶ independently of one another represent hydrogen, hydroxy, C₁₋₄ alkyl or C₁₋₆ alkylthio, or amino substituted or unsubstituted with a substituent selected from the group consisting of C₁₋₆ alkyl, C₁₋₆ hydroxyalkyl and C₁₋₆ aminoalkyl;

R⁷, R⁸, R⁹, R¹⁰ and R¹¹ independently of one another represent hydrogen or C₁₋₆ alkyl, or amino substituted or unsubstituted with a substituent selected from the group consisting of C₁₋₆ alkyl, C₁₋₆ hydroxyalkyl and C₁₋₆ aminoalkyl; and

[symbol] denotes a single bond or a double bond.

The present invention also relates to a process for preparing the compound of formula (I), as defined above, and to an antibacterial composition containing the compound of formula (I) as an active ingredient.

### BACKGROUND ART

Cephalosporin-based antibiotics have been widely used for treatment of infectious diseases caused by pathogenic bacteria in human and animals. They are particularly useful for treatment of diseases caused by bacteria resistant to other antibiotics such as penicillin compounds and for treatment of penicillin-hypersensitive patients. In most of the cases for treating such infectious diseases, it is preferred to use antibiotics showing an antimicrobial activity against both of gram-positive and gram-negative microorganisms. It has been very well known that such antimicrobial activity of cephalosporin antibiotics is largely influenced by the kind of substituents present at 3- or 7-position of cephem ring. Therefore, according to the attempt to develop an antibiotic agent showing a potent antimicrobial activity against broad strains of gram-positive and gram-negative microorganisms numerous cephalosporin antibiotics having various substituents introduced into 3- or 7-position have been developed up to the present.

For instance, British Patent No. 1,399,086 illustrates broadly and generically cephalosporin derivatives represented by the following formula (II):

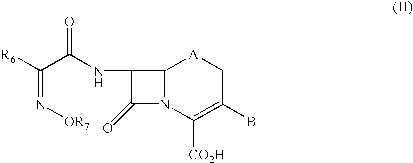

in which

R₆ represents hydrogen or an organic group;

R₇ is an etherified monovalent organic group, which is linked to oxygen via carbon atom;

A represent —S— or >S→O; and

B represents an organic group.

Since development of those compounds, many attempts to develop antibiotic agents having broad antibacterial spectrum have been made and, as a result, numerous cephalosporin antibiotics have been developed. According to their development of them, many studies to introduce acylamido group into 7-position and a certain specific group into C-3 position of the cephem nucleus of formula (II) have also been made in various points of view.

Recently, resistance strains of gram-positive microorganisms, particularly methicillin-resistant Staphylococcus aureus (MRSA) have been recognized as the cause of serious hospital infection and therefore, many attempts have been made to introduce arylthio group into C-3 position to develop cephalosporin compounds showing a potent activity against MRSA.

Thus, Japanese laid-open Pubilcation 98-36375 discloses broadly and generically cephalosporin derivatives represented by the following formula (III) wherein arylthio group is introduced into C-3 position to increase the activity against broad pathogenic strains:

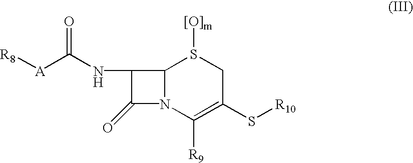

in which

R₈ represents substituted alkylthio, aryl, arylthio, aryloxy or heterocyclyl group;

A represents protected amino, hydroxy or methylene group;

R₉ represents protected carboxy or carboxylate;

R₁₀ represents halo, cyano, amidino, guanidino, azido, nitro, substituted alkyl, alkenyl, dichloroalkyl, aryl, alkoxy, aryloxy, alkylthio, arylthio, alkylamino, acyl, carbamoyl, carbamoyloxy, alkoxyimino, ureido, alkylsulfinyl, alkylsulfonyl or sulfamoyl, or 2-substituted pyrimidinyl, quinazolinyl, purinyl, pyrazolo[3,4-d]pyrimidinyl, pyrazolo[4,3-d]pyrimidinyl, [1,2,3]triazolo[4,5-d]pyrimidinyl or phtheridinyl; and

m denotes 0 or 1.

In the above patent, various heteroaromatic rings are introduced into thioaryl moiety at C-3 position but are different from 4-pyrimidine or 2-pyridine ring used in the present invention. In other words, the above Japanese patent mentions various substituted or unsubstituted pyrimidinyl groups as the substituent present at C-3 position but does not mention 4-pyrimidine or 2-pyridine ring as used in the present invention.

The attempt has been made to develop cephalosporin compounds, which can show a potent activity against serious hospital infection caused by methicillin-resistant Staphylococcus aureus (MRSA), by introducing acyl group into position 7 and pyridine group into C-3 position. Typical example thereof is the compounds of formula (IV) disclosed in European laid-open Publication EP 96-72742:

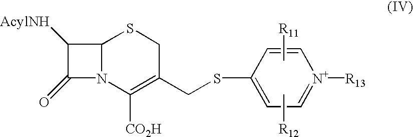

in which

Acyl substituent is Ar—S—CH₂—CO—, wherein Ar represents hydrophobic substituted phenyl, pyridyl or benzthiazolyl group;

R₁₁ and R₁₂ independently of one another represent hydrogen, alkyl or aminoalkylcarbonylamino; and

R₁₃ represents substituted aliphatic, aromatic or arylaliphatic group or a group containing sugar moiety.

In the above European patent, various heteroaromatic rings are introduced into thioaryl moiety present at C-3 position but are different from the substituent present at C-3 position of the compound according to the present invention.

That is, the present invention is characterized by introduction of various substituted or unsubstituted pyrimidinyl or 2-pyridyl groups, which are not disclosed in any of the above patents, into C-3 position.

### DISCLOSURE OF THE INVENTION

Thus, the present inventors have conducted extensive and intensive researches to develop cephalosporin compounds showing broad antibacterial activity against gram-positive microorganisms including MRSA. As a result, we have identified that a certain cephalosporin compound having optionally substituted pyrimidinyl group at C-3 position meets with the above requirement, and then completed the present invention.

Therefore, the purpose of the present invention is to provide a compound of formula (I), as defined above, and pharmaceutically acceptable non-toxic salt, physiologically hydrolyzable ester, hydrate, solvate or isomer thereof.

Further, the purpose of the present invention is to provide a process for preparing the compound of formula (I) and an antibacterial composition containing the compound of formula (I) as an active ingredient.

### BEST MODE FOR CARRYING OUT THE INVENTION

The purpose of the present invention is to provide a novel cephalosporin compound represented by the following formula (I):

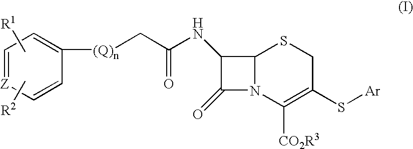

and pharmaceutically acceptable non-toxic salt, physiologically hydrolyzable ester, hydrate, solvate or isomer thereof, in which

R¹ and R² independently of one another represent hydrogen, halogen, C₁₋₆ alkyl, C₁₋₆ alkylthio, aryl, arylthio, or C₅₋₆ heteroaryl containing one or two hetero atoms selected from the group consisting of nitrogen atom and oxygen atom;

R³ represents hydrogen or a carboxy-protecting group;

Q represents O, S or CH₂;

Z represents CH or N;

n denotes an integer of 0 or 1;

Ar represents a heteroaryl group represented by one of the following formulas:

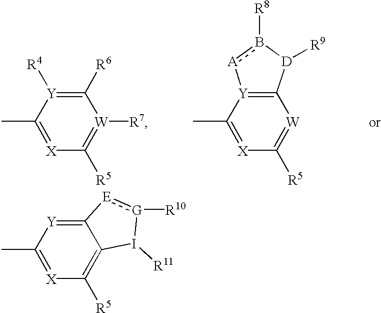

wherein X, Y, W, A, B, D, E, G and I independently of one another represent N or C (or CH), provided that the six-membered ring forms a pyrimidine structure;

R⁴ represents hydrogen or C₁₋₄ alkyl, or amino substituted or unsubstituted with a substituent selected from the group consisting of C₁₋₆ alkyl and C₁₋₆ hydroxyalkyl;

R⁵ and R⁶ independently of one another represent hydrogen, hydroxy, C₁₋₄ alkyl or C₁₋₆ alkylthio, or amino substituted or unsubstituted with a substituent selected from the group consisting of C₁₋₆ alkyl, C₁₋₆ hydroxyalkyl and C₁₋₆ aminoalkyl;

R⁷, R⁸, R⁹, R¹⁰ and R¹¹ independently of one another represent hydrogen or C₁₋₆ alkyl, or amino substituted or unsubstituted with a substituent selected from the group consisting of C₁₋₆ alkyl, C₁₋₆ hydroxyalkyl and C₁₋₆ aminoalkyl; and

[symbol] denotes a single bond or a double bond.

The compound of formula (I) according to the present invention can be administered in the form of an injectable formulation or an oral formulation depending on the purpose of its use.

Pharmaceutically acceptable non-toxic salts of the compound of formula (I) include salts with inorganic acids such as hydrochloric acid, hydrobromic acid, phosphoric acid, sulfuric acid, etc., salts with organic carboxylic acids such as acetic acid, trifluoroacetic acid, citric acid, formic acid, maleic acid, oxalic acid, succinic acid, benzoic acid, tartaric acid, fumaric acid, mandelic acid, ascorbic acid, malic acid, etc., or with methanesulfonic acid or para-toluenesulfonic acid, and salts with other acids which have been well-known and widely used in the technical field of penicillins and cephalosporins. These acid addition salts can be prepared according to any of the conventional methods. Further, the compound of formula (I) can also form a non-toxic salt with a base. The base which can be used for this purpose includes inorganic bases such as alkaline metal hydroxides (e.g. sodium hydroxide, potassium hydroxide, etc.), alkaline metal bicarbonates (e.g. sodium bicarbonate, potassium bicarbonate, etc.), alkaline metal carbonates (e.g. sodium carbonate, potassium carbonate, calcium carbonate, etc.), etc., and organic bases such as amino acids.

Examples of physiologically hydrolysable esters of the compound of formula (I) include indanyl, phthalidyl, methoxymethyl, pivaloyloxymethyl, glycyloxymethyl, phenylglycyloxymethyl, 5-methyl-2-oxo-1,3-dioxolen-4-yl methyl esters or other physiologically hydrolysable esters which have been well-known and widely used in the field of penicillins and cephalosporins. These esters can be prepared according to any of the known conventional methods.

Typical examples of the compound of formula (I) according to the present invention include the following:

I-1: (6R,7R)-3-[(2,6-diamino-4-pyrimidinyl)sulfanyl]-7-({2-[(2,5-dichlorophenyl)-sulfanyl]acetyl}amino)-8-oxo-5-thia-1-azabicyclo[4.2.0]oct-2-ene-2-carboxylic acid,

I-2: (6R,7R)-3-[(2-amino-6-hydroxy-4-pyrimidinyl)sulfanyl]-7-({2-[(2,5-dichloro-phenyl)sulfanyl]acetyl}amino)-8-oxo-5-thia-1-azabicyclo[4.2.0]oct-2-ene-2-carboxylic acid,

I-3: (6R,7R)-3-[(6-amino-2-hydroxy-4-pyrimidinyl)sulfanyl]-7-({2-[(2,6-dichloro-phenyl)sulfanyl]acetyl}amino)-8-oxo-5-thia-1-azabicyclo[4.2.0]oct-2-ene-2-carboxylic acid,

I-4: (6R,7R)-3-[(4-amino-2-pyrimidinyl)sulfanyl]-8-oxo-7-[(phenylacetyl)amino]-5-thia-1-azabicyclo[4.2.0]oct-2-ene-2-carboxylic acid,

I-5: (6R,7R)-3-[(2,6-diamino-4-pyrimidinyl)sulfanyl]-7-({2-[(2,6-dichloro-4-pyridinyl)sulfanyl]acetyl}amino)-8-oxo-5-thia-1-azabicyclo[4.2.0]oct-2-ene-2-carboxylic acid, and

I-6: (6R,7R)-3-[(2-amino-6-hydroxy-4-pyrimidinyl)sulfanyl]-7-({2-[(2,6-dichloro-4-pyridinyl)sulfanyl]acetyl}amino)-8-oxo-5-thia-1-azabicyclo[4.2.0]oct-2-ene-2-carboxylic acid.

According to the present invention, the compound of formula (I):

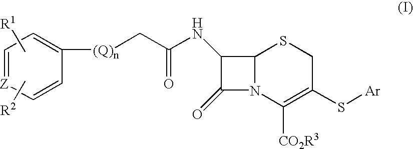

wherein R¹, R², R³, Z, Q, n and Ar are as defined above, and pharmaceutically acceptable non-toxic salt, physiologically hydrolysable ester, hydrate, solvate or isomer thereof can be prepared by a process which comprises reacting a compound of formula (V):

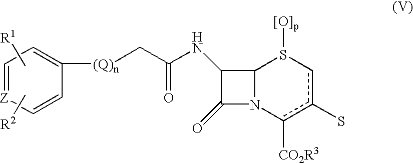

wherein R¹, R², R³, Z, Q and n are as defined in the formula (I), L represents a leaving group and p is 0 or 1, with a compound of formula (VI):

HS—Ar (VI)

wherein Ar is as defined in the formula (I), in a solvent and, if necessary, removing the acid-protecting group before or after the reaction, or reducing S→oxide of a compound of formula (VII)

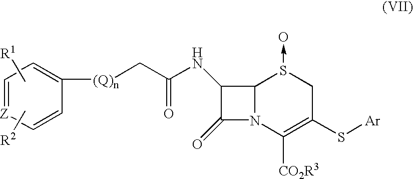

wherein R¹, R², R³, Z, Q, n and Ar are as defined in the formula (I).

In the process for preparing the compound of formula (I) according to the present invention, the compound of formula (VI) is used in an amount of 1 to 2 moles with respect to one mole of the compound of formula (V).

In the process for preparing the compound of formula (I) by reacting the compound of formula (V) with the compound of formula (VI) according to the present invention, the reaction temperature can be varied within a broad range and is generally in the range of −10° C. to 50° C., preferably in the range of 20° C. to 35° C.

The process for preparing the compound of formula (I) according to the present invention can be carried out using a solvent. Suitable solvent for this purpose is a non-reactive solvent and includes, for example, dimethylformamide, dimethylsulfoxide, methylene chloride, etc., or the mixture thereof.

In the above process, carboxy-protecting group R³ is desirably the group which can be readily removed under mild condition. Typical examples of carboxy-protecting group R³ include (lower) alkyl ester (e.g. methyl ester, t-butyl ester, etc.), (lower) alkenyl ester (e.g. vinyl ester, allyl ester, etc.), (lower) alkylthio (lower) alkyl ester (e.g. methylthiomethyl ester, etc.), halo (lower) alkyl ester (e.g. 2,2,2-trichloroethyl ester, etc.), substituted or unsubstituted aralkyl ester (e.g. benzyl ester, p-nitrobenzyl ester, p-methoxybenzyl ester, etc.) or silyl ester. These carboxy-protecting groups can be readily removed under mild reaction conditions such as hydrolysis, reduction, etc. to generate a free carboxy group, and appropriately selected depending on the chemical properties of the compound of formula (I).

A suitable leaving group L is, for example, methanesulfonyloxy group, trifluoromethanesulfonyloxy group, etc. (see, Synthesis of its precursor 3-hydroxy compounds: Hel. Chem. Acta 1974, 57, 1919-1934).

The dotted line in the formula (V) represents each of 2-cephem and 3-cephem compounds, as shown in the following, or their mixture:

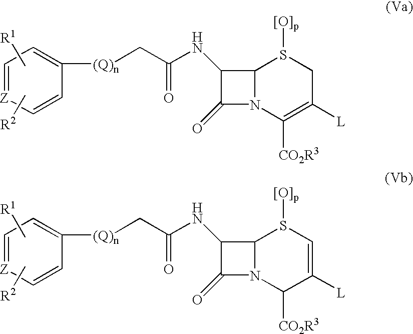

wherein R¹, R², R³, Z, Q, n, p and L are as defined above.

The compound of formula (V) can be prepared by activating a compound of formula (VIII):

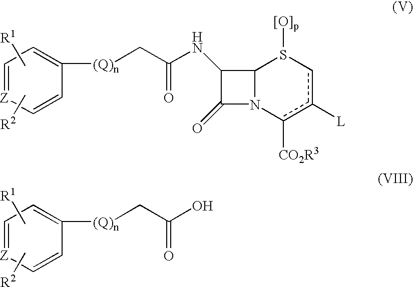

wherein R¹, R², R³, Z, Q, n, p and L are as defined above, or its salt with an acylating agent and then reacting the resulting activated compound with a compound of formula (IX):

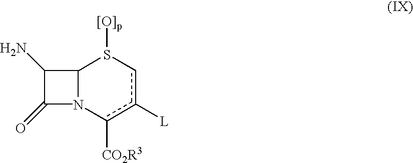

wherein R³, p and L are as defined above.

The dotted line in the formula (IX) represents each of 2-cephem and 3-cephem compounds, as shown in the following, or their mixture:

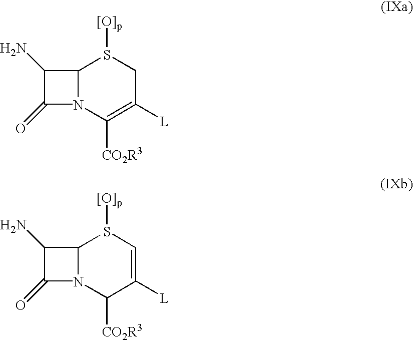

wherein R³, p and L are as defined above.

The compound of formula (I) can also prepared by reacting the compound of formula (VI):

HS—Ar (VI)

wherein Ar is as defined above, with a compound of formula (X):

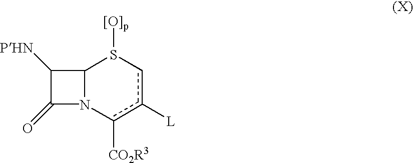

wherein R³, p and L are as defined above and P′ represents an amino-protecting group, to provide a compound of formula (XI):

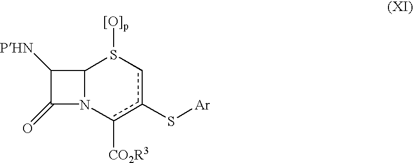

wherein R³, P′, Ar and p are as defined above; removing the amino-protecting group P′ from the compound of formula (XI), activating a carboxylic acid of formula (VIII) or its salt with an acylating agent, and then reacting the activated form of the compound of formula (VIII) with the deprotected compound of formula (XI) from which protecting group P′ is removed (see, Preparation of activated carboxylic acids and reaction of activated carboxylic acid with amine group: EP 94105499.1, EP 94108809.8).

In the above process for preparing the compound of formula (I), the compound of formula (X) is used generally in an amount of 0.5 to 2 moles with respect to one mole of the compound of formula (VI) and the compound of formula (VIII) is used generally in an amount of 1 to 2 moles with respect to one mole of the compound of formula (XI).

In reacting the compound of formula (VI) with the compound of formula (X), the reaction temperature can be varied within a broad range and is generally in the range of −80° C. to 40° C. This reaction can be carried out in a solvent. Suitable solvent which can be used in this reaction is an inert solvent and includes, for example, tetrahydrofuran, dimethylformamide, dimethylsulfoxide, methylene chloride and the mixture thereof.

The dotted line in the formula (X) represents each of 2-cephem and 3-cephem compounds, as shown in the following, or their mixture

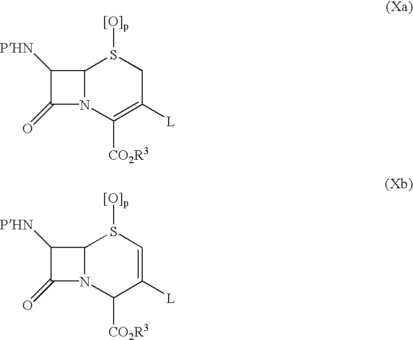

wherein R³, p, L and P′ are as defined above.

In preparing the compound of formula (V), an acylated derivative as the activated form of the compound of formula (VIII) includes acid chlorides, acid anhydrides, mixed acid anhydrides (preferably, acid anhydrides formed with methylchloroformate, mesitylenesulfonyl chloride, p-toluenesulfonyl chloride or chlorophosphate) or activated esters (preferably, esters formed from the reaction with N-hydroxybenzotriazole in the presence of a condensing agent such as dicyclohexylcarbodiimide), etc. In addition, the acylation reaction can also be practiced by using a free acid compound of formula (VIII) in the presence of a condensing agent such as dicyclohexylcarbodiimide or carbonyldiimidazole. Further, the acylation reaction is well practiced generally in the presence of an organic base, preferably a tertiary amine such as triethylamine, dimethylaniline, pyridine, etc., or an inorganic base such as sodium bicarbonate, sodium carbonate, etc. The solvent which can be used in this reaction includes halogenated hydrocarbon such as methylene chloride, chloroform, etc., tetrahydrofuran, acetonitrile, dimethylformamide or dimethyl acetamide. The mixed solvent comprising two or more solvents selected from the above can also be used. The reaction can also be carried out in an aqueous solution.

The reaction temperature in the acylation reaction is in the range of −50° C. to 50° C., preferably in the range of −30° C. to 20° C. The acylating agent for the compound of formula (VIII) can be used in an equimolar amount or a slightly excessive amount, i.e. in an amount of 1.05 to 1.2 equivalent weights, with respect to an equivalent weight of the compound of formula (IX) or (X).

In preparing the compound of formula (I) as defined above, the amino-protecting group or the acid-protecting group present in the compound of formula (V) can be removed by any of the conventional methods widely known in the field of cephalosporins. That is, the protecting groups can be removed by hydrolysis or reduction. Acid hydrolysis is useful for removing tri(di)phenylmethyl group or alkoxycarbonyl group and is carried out using an organic acid such as formic acid, trifluoroacetic acid, p-toluenesulfonic acid, etc., or an inorganic acid such as hydrochloric acid, etc.

The resulting product from the above processes can be treated with various methods such as recrystallization, electrophoresis, silica gel column chromatography or ion exchange resin chromatography to separate and purify the desired compound of formula (I).

Another purpose of the present invention is to provide a pharmaceutical composition containing the compound of formula (I) or its pharmaceutically acceptable salt as an active ingredient, together with a pharmaceutically acceptable carrier.

The compound according to the present invention can be administered in the form of an injectable formulation or an oral formulation depending on the purpose of its use.

The compound of formula (I) of the present invention can be formulated using known pharmaceutically acceptable carriers and excipients according to the known method to prepare a unit dosage form or to be introduced into a multi-dosage container. The formulations can be in the form of a solution, suspension or emulsion in an oil or aqueous medium and can contain conventional dispersing agent, suspending agent or stabilizing agent. In addition, the formulation can also be in the form of a ready-to-use dry powder which can be used by dissolving with a sterile, pyrogen-free water before its use. The compound of formula (I) can also be formulated in the form of a suppository by using conventional suppository bases such as cocoa butter or other glycerides. Solid dosage form for oral administration includes capsules, tablets, pills, powders and granules, with capsules and tablets being particularly useful. For the tablets and pills, it is preferred to provide an enteric coating. Solid dosage form can be prepared by mixing the active compound of formula (I) according to the present invention with one or more inert diluents such as sucrose, lactose, starch, etc., and carriers including lubricants such as magnesium stearate, disintegrating agents, binders, etc.

If necessary, the compound of the present invention can be administered in combination with other antibacterial agent such as penicillins or other cephalosporins.

In formulating the compound of formula (I) according to the present invention into the unit dosage form, it is preferred that the unit dosage form contains the active ingredient of formula (I) in an amount of about 50 to 1,500 mg. The dosage of the compound of formula (I) is sutably selected under the physician's prescription depending on various factors including weight and age of patient, particular conditions and severity of diseases to be treated, etc. However, the daily dosage for treatment of adult man generally corresponds to about 500 to 5,000 mg of the compound of formula (I) depending on the frequency and intensity of administration. For intramuscular or intravenous injection to adult man, a total daily dosage in the range of about 150 to 3,000 mg is generally sufficient. However, in case of infections caused by some pathogenic strains, it may be preferred to more increase the daily doage.

The compound of formula (I) and its non-toxic salt, preferably salts with alkali metals, alkaline earth metals, inorganic acids, organic acids and amino acids, according to the present invention exhibit a potent antimicrobial activity and a broad antibacterial spectrum against broad pathogenic microorganisms including various gram-positive strains and therefore, are very useful for prevention and treatment of diseases caused by bacterial infection in animals including human being.

The present invention will be more specifically illustrated by the following preparations and examples. However, it should be understood that these preparations and examples are provided only to help clear understanding of the present invention but do not intend to limit the present invention in any manner.

### EXAMPLES

### Preparation 1

### Synthesis of benzhydryl 7-amino-3-[(2,6-diamino-4-pyrimidinyl)sulfanyl]-8-oxo-5-thia-1-azabicyclo [4.2.0]oct-2-ene-2-carboxylate

2 g (4.025 mmol) of (6R,7R)-benzhydryl 7-amino-3-[(methoxysulfanyl)oxy]-8-oxo-5-thia-1-azabicyclo[4.2.0]oct-2-ene-2-carboxylate hydrochloride was dissolved in 4/10 ml of tetrahydrofuran/dimethylformamide, and the temperature of the reaction vessel was then lowered to −78° C. 0.731 mg (3.823 mmol) of 2,4-diamino-6-mercapto pyrimidine ½ sufate was added to the reaction solution. The reaction mixture was stirred for 24 hours while gradually increasing the reaction temperature to room temperature. Excessive ether was added to solidify the resulting product, which was filtered and then dried under nitrogen to obtain 2.23 g (yield 85%) of the title compound.

¹H-NMR (CD₃OD) δ7.36−7.30 (10H, m), 7.01 (1H, s), 5.91 (1H, s), 5.54−5.52 (1H, d, J=5.5 Hz), 5.28−5.27 (1H, d, J=5.5 Hz), 4.05−4.01 (1H, Abq, J=17.9 Hz), 3.70−3.67 (1H, Abq, J=18.3 Hz).

Mass (m/e) 376

### Example 1

### Synthesis of (6R,7R)-3-[(2,6-diamino-4-pyrimidinyl)sulfanyl]-7-({2-[(2,5-dichloro-phenyl)sulfanyl]acetyl}amino)-8-oxo-5-thia-1-azabicyclo[4.2.0]oct-2-ene-2-carboxylic acid

2.23 g of benzhydryl 7-amino-3-[(2,6-diamino-4-pyrimidinyl)sulfanyl]-8-oxo-5-thia-1-azabicyclo[4.2.0]oct-2-ene-2-carboxylate was treated with trifluoroacetic acid and anisole to obtain 1.8 g of deprotected trifluoroacetate compound. 0.5 g (1.1012 mmol) of the resulting salt compound was dissolved in 5 ml of dichloromethane and then cooled to 0° C. 1.1 ml of N,O-bistrimethylsilyl acetamide (BSA) was added and the mixture was stirred at 0° C. for 10 minutes. To the reaction mixture were added 0.31 g (1.12113 mmol) of 2,5-dichlorophenylthioacetyl chloride and 0.045 ml (0.5506 mmol) of pyridine. The temperature of the reaction vessel was increased to 10° C. and stirred for 2 hours. The reaction was stopped with water and ammonium chloride. The resulting product was solidified with diethyl ether to obtain 0.28 g of the solid product, which was then purified with a high pressure fractional liquid chromatography to obtain 0.080 g (yield 14.5%) of the title compound.

¹H-NMR (DMSO-d₆) δ9.34 (1H, d, J=7.8 Hz), 7.48−7.46 (2H, m), 7.26−7.24 (1H, m), 6.65 (1H, br, s), 6.34 (1H, br, s), 5.76−5.67 (2H, m, s), 5.19−5.18 (1H, d, J=4.55 Hz), 3.98 (2H, s).

Mass (m/e) 558

### Example 2

### Synthesis of (6R,7R)-3-[(2-amino-6-hydroxy-4-pyrimidinyl)sulfanyl]-7-({2-[(2,5-dichlorophenyl)sulfanyl]acetyl}amino)-8-oxo-5-thia-1-azabicyclo[4.2.0]oct-2-ene-2-carboxylic acid

0.085 g (yield 33.3%) of the title compound was obtained according to the same procedure as Example 1.

¹H-NMR (CD₃OD) δ8.89 (1H, m), 7.46 (1R s), 7.37−7.35 (1H d, J=8.25 Hz), 7.16 (1H, m), 5.78−5.77 (2H, s, d), 5.25−5.24 (1H, d, J=5 Hz), 3.88 (1H, Abq, J=17.9 Hz), 3.82 (2H, s), 3.51 (1H, Abq, J=17.9 Hz).

Mass (m/e) 559

### Example 3

### Synthesis of (6R,7R)-3-[(6-amino-2-hydroxy-4-pyrimidinyl)sulfanyl]-7-({2-[(2,5-dichlorophenyl)sulfanyl]acetyl}amino)-8-oxo-5-thia-1-azabicyclo[4.2.0]oct-2-ene-2-carboxylic acid

0.07 g (yield 30.0%) of the title compound was obtained according to the same procedure as Example 1.

¹H-NMR (CD₃OD) δ8.91−8.89 (1H, m), 7.45 (1H, s), 7.37−7.35 (1H, d, J=8.25 Hz), 7.19 (1H, m), 5.93 (1H, s), 5.72−5.71 (1H, d, J=5 Hz), 5.29−5.27 (1H, d, J=5.05 Hz), 3.94−3.91 (1H, Abq, J=17.9 Hz), 3.86 (2H, s), 3.52−3.50 (1H, Abq, J=17.9 Hz).

Mass (m/e) 559

### Example 4

### Synthesis of (6R,7R)-3-[(4-amino-2-pyrimidinyl)sulfanyl]-8-oxo-7-[(phenylacetyl)-amino]-5-thia-1-azabicyclo[4.2.0]oct-2-ene-2-carboxylic acid

1.0 g of benzhydryl (6R,7R)-3-hydroxy-8-oxo-7-[(2-phenylacetyl)amino]-5-thia-1-azabicyclo[4.2.0]oct-2-ene-2-carboxylate was dissolved in dichloromethane solution and then cooled to −78° C. 0.336 ml of trifluoromethanesulfonic acid anhydride and 0.174 ml of diisopropylethylamine were successively added dropwise to the reaction solution. The mixture was stirred for 1.5 hours and then, excessive dichloromethane was poured. The solution was washed with water and saline, dried over magnesium sulfate, filtered and then concentrated under reduced pressure. Without further separation, the subsequent reaction was conducted.

The resulting triflate and 380 mg of 2-amino-2-pyrimidine thiol were dissolved in 15 ml of dimethylformamide and then allowed to initiate the reaction at the temperature of −20° C. The temperature was slowly increased to room temperature and then, the reaction mixture was stirred for 12 hours. The reaction solution was diluted with ethyl acetate, washed with saline, dried over anhydrous magnesium sulfate and then filtered. The filtrate was distilled under reduced pressure. The residue was treated with dichloromethane and diethyl ether to precipiate the crystal, which was then filtered to obtain benzhydryl (6R,7R)-3-[(4-amino-2-pyrimidinyl)sulfanyl]-8-oxo-7-({2-[4-pyridi nyl)sulfanyl]acetyl}amino)-5-thia-1-azabicyclo[4.2.0]oct-2-ene-2-carboxylate.

The resulting solid was deprotected with 2 ml of trifluoroacetic acid and 0.8 ml of anisole and then, separated and purified with a high pressure fractional liquid chromatography to obtain 30 mg (yield through three steps 3.4%) of the title compound.

¹H-NMR (D₂O) δ7.95 (1H, d, J=5.96 Hz), 7.40-7.47 (5H, m), 6.40 (1H, d, J=5.96 Hz), 5.94 (1H, d, J=4.58 Hz), 5.06 (1H, d, J=4.58 Hz), 4.04 (2H, s), 3.79 (2H, Abq, J=15.12 Hz).

Mass (m/e) 444

### Example 5

### Synthesis of (6R,7R)-3-[(2,6-diamino-4-pyrimidinyl)sulfanyl]-7-({2-[(2,6-dichloro-4-pyridinyl)sulfanyl]acetyl}amino)-8-oxo-5-thia-1-azabicyclo [4.2.0oct-2-ene-2-carboxylic acid

2.23 g of benzhydryl 7-amino-3-[(2,6-diamino-4-pyrimidinyl)sulfanyl]-8-oxo-5-thia-1-azabicyclo[4.2.0]oct-2-ene-2-carboxylate was treated with trifluoroacetic acid and anisole to obtain 1.8 g of deprotected trifluoroacetate compound.

0.20 g (0.4415 mmol) of the resulting (6R,7R)-7-amino-3-[(2,6-diamino-4-pyrimidinyl)sulfanyl]-8-oxo-5-thia-1-azabicyclo[4.2.0]oct-2-ene-2-carboxylic acid trifluoroacetate was dissolved in 3 ml of dichloromethane and then cooled to 0° C. 0.44 ml of N,O-bistrimethylsilyl acetamide was added and the reaction mixture was stirred at 0° C. for 10 minutes. 0.14 g (0.5298 mmol) of 2-[(2,6-dichloro-4-pyridinyl)-sulfanyl]acetyl chloride and 0.018 ml (0.2208 mmol) of pyridine were added. The temperature of the reaction vessel was increased to 10° C. and the reaction solution was stirred for 3 hours. The reaction was stopped with water and ammonium chloride. The resulting product was solidified with diethyl ether to obtain 0.10 g of the solid product, which was then purified with a high pressure fractional liquid chromatography to obtain 0.038 g (yield 15.4%) of the title compound.

¹H-NMR (D₂O) δ7.14 (1H, s), 5.58 (1H, s), 5.40−5.39 (1H, d, J=4.4 Hz), 5.01−4.99 (1H, d, J=4.8 Hz), 3.83 (2H, s), 3.58−3.54 (1H, ABq, J=17.6 Hz), 3.17−3.13 (1H, Abq, J=17.2 Hz).

Mass (m/e) 559

### Example 6

### Synthesis of (6R,7R)-3-[(2-amino-6-hydroxy-4-pyrimidinyl)sulfanyl]-7-({2-[(2,6-dichloro-4-pyridinyl)sulfanyl]acetyl}amino)-8-oxo-5-thia-1-azabicyclo[4.2.0]oct-2-ene-2-carboxylic acid

0.20 g (0.4415 mmol) of (6R,7R)-7-amino-3-[(2-amino-6-hydroxy-4-pyrimidinyl)sulfanyl]-8-oxo-5-thia-1-azabicyclo[4.2.0]oct-2-ene-2-carboxylic acid trifluo roacetate was dissolved in 3 ml of dichloromethane and then cooled to 0° C. 0.44 ml of N,O-bistrimethylsilyl acetamide was added and the reaction mixture was stirred at 0° C. for 10 minutes. 0.14 g (0.5298 mmol) of 2-[(2,6-dichloro-4-pyridinyl)-sulfanyl]acetyl chloride and 0.018 ml (0.2208 mmol) of pyridine were added. The temperature of the reaction vessel was increased to 10° C. and the reaction solution was stirred for 3 hours. The reaction was stopped with water and ammonium chloride. The resulting product was solidified with diethyl ether to obtain 0.15 g of the solid product, which was then purified with a high pressure fractional liquid chromatography to obtain 0.077 g (yield 31.1%) of the title compound.

¹H-NMR (D₂O) β7.02 (2H, s), 5.32−5.30 (1H, d, J=4.4 Hz), 5.26 (1H, s), 4.88−4.87 (1H, d, J=4.8 Hz), 3.71−3.68 (2H, q), 3.46−3.42 (1H, ABq, J=17.6 Hz), 3.05−3.01 (1H, Abq, J=16.8 Hz).

Mass (m/e) 560

### Experiment 1: Minimum Inhibitory Concentration (MIC)

The effectiveness of the compound according to the present invention was determined by obtaining Minimum Inhibitory Concentration (MIC) of the compounds prepared by the above examples (I-1 to I-6) and vancomycin, which is the known compound having a potent activity against gram-positive strains, as the control drug against the standard strains. Specifically, Minimum Inhibitory Concentration was obtained by diluting the test material with a double dilution method, dispersing them in Mueller-Hinton agar medium, inoculating each of the test strain having 10⁷ cfu (colony forming unit) per ml in an amount of 2 μl to the medium and then incubating them at 37° C. for 20 hours. The results are shown in the following Tables 1 and 2. From the result of Minimum Inhibitory Concentration test, it can be seen that the compound according to the present invention has a good activity against major pathogenic microorganisms, which cause hospital infection, including MRSA strains.

TABLE 1
Sensitivity test result using standard strains (μg/ml)

| | | S. | S. | S. | E. |
|---|---|---|---|---|---|
| | Staphylococcus | aureus | aureus | epidermidis | faecalis |
| | aureus giorgio | 77 | 241 | R005 | L239 |
| I-1 | <0.008 | 0.25 | 2 | 0.25 | 2 |
| I-2 | 0.031 | 0.5 | 4 | 0.5 | 2 |
| I-3 | 0.016 | 1 | 4 | 0.5 | 2 |
| I-4 | 4 | 32 | >64 | 32 | >64 |
| Vancomycin | 1 | 1 | 2 | 1 | 2 |

TABLE 2
Sensitivity test result using standard strains (μg/ml)

| | Staphylococcus | S. aureus | Staphylococcus | S. epidermidis | E. faecalis |
|---|---|---|---|---|---|
| | aureus giorgio | 77 | aureus K311 | R005 | EFS004 |
| I-5 | 0.063 | 1 | 1 | 1 | 4 |
| I-6 | 0.063 | 2 | 1 | 0.5 | 2 |
| Vancomycin | 1 | 1 | 2 | 1 | 1 |

While the invention has been described with respect to the above specific embodiments, it should be recognized that various modifications and changes can be made to the invention by those skilled in the art, which also fall within the scope of the invention as defined by the appended claims.

1. A cephalosporin compound represented by the following formula (I): 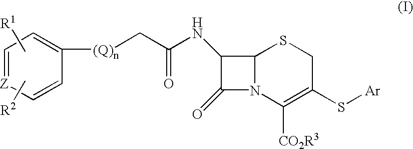
and pharmaceutically acceptable non-toxic salt, physiologically hydrolyzable ester, hydrate, solvate or isomer thereof, in which
R¹ and R² independently of one another represent hydrogen, halogen, C₁₋₆ alkyl, C₁₋₆ alkylthio, aryl, arylthio, or C₅₋₆ heteroaryl containing one or two hetero atoms selected from the group consisting of nitrogen atom and oxygen atom;
R³ represents hydrogen or a carboxy-protecting group;
Q represents O, S or CH₂;
Z represents CH or N;
n denotes an integer of 0 or 1;
Ar represents a heteroaryl group represented by one of the following formulas: 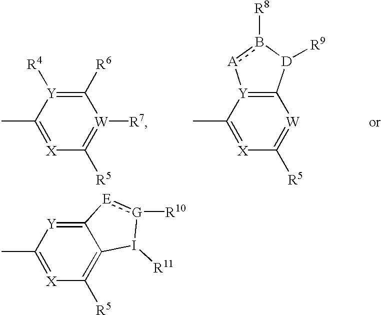
wherein X, Y, W, A, B, D, E, G and I independently of one another represent N or C (or CH), provided that the six-membered ring forms a pyrimidine structure;
R⁴ represents hydrogen or C₁₋₄ alkyl, or amino substituted or unsubstituted with a substituent selected from the group consisting of C₁₋₆ alkyl and C₁₋₆ hydroxyalkyl;
R⁵ and R⁶ independently of one another represent hydrogen, hydroxy, C₁₋₄ alkyl or C₁₋₆ alkylthio, or amino substituted or unsubstituted with a substituent selected from the group consisting of C₁₋₆ alkyl, C₁₋₆ hydroxyalkyl and C₁₋₆ aminoalkyl;
R⁷, R⁸, R⁹, R¹⁰ and R¹¹ independently of one another represent hydrogen or C₁₋₆ alkyl, or amino substituted or unsubstituted with a substituent selected from the group consisting of C₁₋₆ alkyl, C₁₋₆ hydroxyalkyl and C₁₋₆ aminoalkyl; and [symbol]
denotes a single bond or a double bond.

2. The compound of claim 1, wherein the compound is selected from the group consisting of the following:
(6R,7R)-3-[(2,6-diamino-4-pyrimidinyl)sulfanyl]-7-({2-[(2,5-dichlorophenyl)-sulfanyl]acetyl}amino)-8-oxo-5-thia-1-azabicyclo[4.2.0]oct-2-ene-2-carboxylic acid,
(6R,7R)-3-[(2-amino-6-hydroxy-4-pyrimidinyl)sulfanyl]-7-({2-[(2,5-dichloro-phenyl)sulfanyl]acetyl}amino)-8-oxo-5-thia-1-azabicyclo[4.2.0]oct-2-ene-2-carboxylic acid,
(6R,7R)-3-[(6-amino-2-hydroxy-4-pyrimidinyl)sulfanyl]-7-({2-[(2,6-dichloro-phenyl)sulfanyl]acetyl}amino)-8-oxo-5-thia-1-azabicyclo[4.2.0]oct-2-ene-2-carboxylic acid,
(6R,7R)-3-[(4-amino-2-pyrimidinyl)sulfanyl]-8-oxo-7-[phenylacetyl)amino]-5-thia-1-azabicyclo[4.2.0]oct-2-ene-2-carboxylic acid,
(6R,7R)-3-[(2,6-diamino-4-pyrimidinyl)sulfanyl]-7-({2-[(2,6-dichloro-4-pyridinyl)sulfanyl]acetyl}amino)-8-oxo-5-thia-1-azabicyclo[4.2.0]oct-2-ene-2-carboxylic acid, and
(6R,7R)-3-[(2-amino-6-hydroxy-4-pyrimidinyl)sulfanyl]-7-({2-[(2,6-dichloro-4-pyridinyl)sulfanyl]acetyl}amino)-8-oxo-5-thia-1-azabicyclo[4.2.0]oct-2-ene-2-carboxylic acid.

3. A process for preparing the compound of formula (I) according to claim 1, which comprises reacting a compound of formula (V): 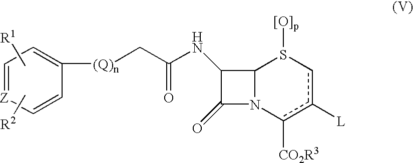
wherein R¹, R², R³, Z, Q and n are as defined in claim 1, L represents a leaving group and p is 0 or 1, with a compound of formula (VI):
HS—Ar (VI)
wherein Ar is as defined in claim 1, in a solvent or reducing S→oxide of a compound of formula (VII): 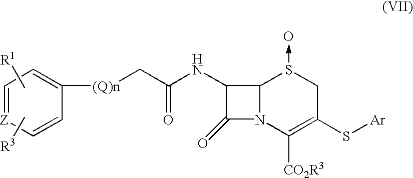
wherein R¹, R², R³, Z, Q, n and Ar are as defined in claim 1.

4. The process of claim 3, which further comprises removing acid-protecting group.

5. A process for preparing the compound of formula (I) according to claim 1, which comprises reacting the compound of formula (VI):
HS—Ar (V)
wherein Ar is as defined in claim 1, with a compound of formula (X): 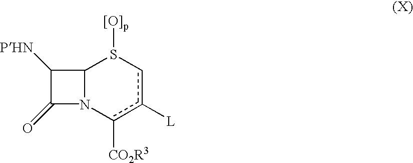
wherein R³ is as defined in claim 1, p is 0 or 1, L is a leaving group and P′ represents an amino-protecting group, to provide a compound of formula (XI): 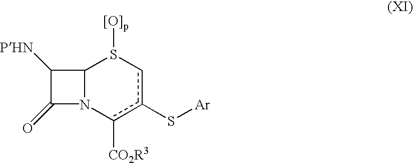
wherein R³, P′, Ar and p are as defined above; removing the amino-protecting group P′ from the compound of formula (XI), activating a carboxylic acid of formula (VII) or its salt with an acylating agent, and then reacting the activated form of the compound of formula (VIII) with the deprotected compound of formula (XI) from which protecting group P′ is removed.

6. An antibacterial composition containing the compound of formula (I) or its pharmaceutically acceptable salt according to claim 1 as an active ingredient, together with a pharmaceutically acceptable carrier.
